(19) United States
(12) Patent Application Publication
(10) Pub. No.: US 2015/0118548 A1
(43) Pub. Date: Apr. 30, 2015

(54) ELECTROLYTIC SOLUTION, METHOD FOR PREPARING ESTER COMPOUND CONTAINED THEREIN AND LITHIUM SECONDARY CELL

(71) Applicant: NEC Corporation, Tokyo (JP)

(72) Inventors: Masahiro Suguro, Tokyo (JP); Midori Shimura, Tokyo (JP)

(73) Assignee: NEC Corporation, Tokyo (JP)

(21) Appl. No.: 14/405,382

(22) PCT Filed: May 28, 2013
(86) PCT No.: PCT/JP2013/064752

§ 371 Date: Dec. 3, 2014

(30) Foreign Application Priority Data
Jun. 6, 2012 (JP) 2012-128833

(51) Int. Cl. H01M 10/0567; H01M 4/133; H01M 4/134; C07C 227/10; H01M 4/587; C07C 67/31; C07D 295/145; H01M 10/0525; H01M 4/38

(52) U.S. Cl. CPC H01M 10/0567; H01M 10/0525; H01M 4/133; H01M 4/134; H01M 4/386; H01M 4/587; C07C 67/31; C07D 295/145; C07C 227/10; H01M 2004/028
USPC 429/188; 560/181; 546/248; 560/171

(57) ABSTRACT

Provided is a lithium secondary cell which has high capacity, suppresses deterioration in capacity and improves cycle characteristics particularly when used in high-temperature environments and has long lifespan. Provided is a lithium secondary cell including a positive electrode active material layer containing a positive electrode active material, a negative electrode active material layer containing a negative electrode active material and an electrolytic solution for immersing the positive and negative electrode active material layers, wherein the electrolytic solution contains at least one certain ester compound.

### TECHNICAL FIELD

The present invention relates to a lithium secondary cell having high capacity, superior cycle characteristics particularly when used in high-temperature environments and long lifespan, an electrolytic solution used for the lithium secondary cell and a method for preparing an ester compound contained therein.

### BACKGROUND

Lithium secondary cells are widely used for portable electronic devices, personal computers and the like and required miniaturization and weight lightening, while required high energy density, suppression of deterioration upon charge/discharge, superior cycle characteristics and long lifespan for highly functional electronic devices, electric vehicles and the like. Lithium cells have a configuration in which a positive electrode active material layer containing a positive electrode active material and a negative electrode active material layer containing a negative electrode active material respectively formed on current collectors face each other via a separator interposed therebetween, are immersed in an electrolytic solution and accommodated in an outer package, and the electrode active materials reversibly intercalate and deintercalate lithium ions during charge/discharge cycles.

As such a negative electrode active material, silicon or silicon oxide, a metal such as tin forming an alloy with lithium or oxide of the metal is used instead of a carbon-based material in terms of high energy density, low cost and safety. However, the negative electrode active material layer containing silicon greatly expands and contracts in volume upon charge/discharge and products formed by reaction with the electrolytic solution upon repeated charge/discharge are detached as fine powders from the negative electrode active material layer, thus causing deterioration in cell capacity. Cells using silicon or silicon oxide as the negative electrode active material undergo great capacity deterioration when used under high-temperature environments of 45° C. or higher and such deterioration is serious in the case of stacked laminate-type cells.

In order to suppress deterioration involved in charge/discharge, a negative electrode containing carbon material particles, silicon particles and silicon oxide particles as negative electrode active materials (Patent Document 1) and a negative electrode using particles having a carbon film on the surface of silicon dioxide particles in which silicon is dispersed (Patent Document 2), and the like are reported.

Meanwhile, improvement in cycle characteristics is performed by adding a certain material to an electrolytic solution. A cell using an electrolytic solution containing, as the added material, specifically, cyclic acid anhydride such as succinic anhydride, glutaric anhydride or maleic anhydride and cyclic ester carbonate derivatives having a halogen atom (Patent Document 3), a secondary cell that prevents overcharge using an electrolytic solution containing dicarboxylic acid diester such as dicarboxylic acid dialkylester (Patent Document 4), and the like are reported. There is a need for lithium secondary cells which have greatly increased capacity, suppress deterioration in capacity under use upon high-temperature environments, improve cycle characteristics and have long lifespan.

### PRIOR ART DOCUMENTS

### Patent Documents

Patent Document 1: JP Patent Application Publication No. 2003-123740

Patent Document 2: JP Patent Application Publication No. 2004-47404

Patent Document 3: JP Patent Application Publication No. 2006-294373

Patent Document 4: JP Patent Application Publication No. 2002-367673

### SUMMARY OF THE INVENTION

An object of the present invention is to provide an electrolytic solution for a lithium secondary cell which has high capacity, suppresses deterioration in capacity and improves cycle characteristics particularly when used in high-temperature environments and has long lifespan, a lithium secondary cell using the same and a method for preparing an ester compound contained in the electrolytic solution.

The present invention relates to an electrolytic solution containing an ester compound represented by the following Formula (1):

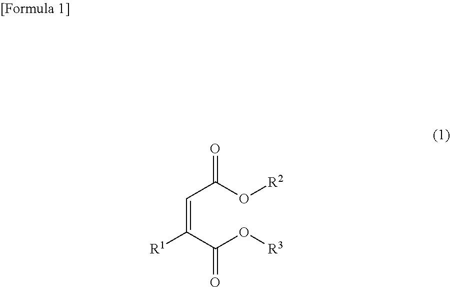

wherein R¹ represents a C2-C12 alkoxy group which may have a substituent, or a C2-C12 alkylamino group which may have a substituent, and R² and R³ independently represent a hydrogen atom or a C2-C12 alkyl group which may have a substituent.

In addition, the present invention relates to a lithium secondary cell comprising a positive electrode active material layer containing a positive electrode active material, a negative electrode active material layer containing a negative electrode active material and an electrolytic solution for immersing the same, wherein the electrolytic solution is an ester compound represented by Formula (1).

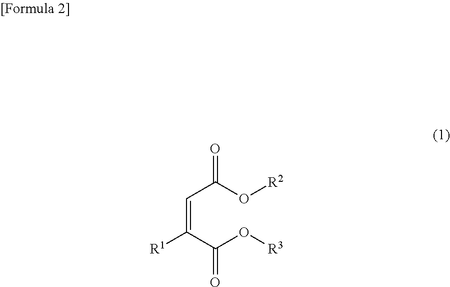

wherein R¹ represents a C2-C12 alkoxy group which may have a substituent, or a C2-C12 alkylamino group which may have a substituent, and R² and R³ independently represent a hydrogen atom or a C2-C12 alkyl group which may have a substituent.

In addition, the present invention relates to a method for preparing an ester compound including reacting an active proton compound represented Formula (2) with an acetylenedicarboxylic acid diester represented by the following Formula (3) to preparing an ester compound represented by Formula (1):

R¹—H (2)

wherein R¹ represents a C2-C12 alkoxy group which may have a substituent, or a C2-C12 alkylamino group which may have a substituent;

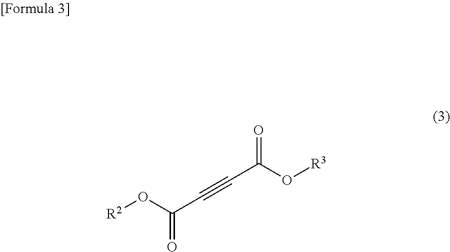

wherein R² and R³ independently represent a hydrogen atom, or a C2-C12 alkyl group which may have a substituent; and

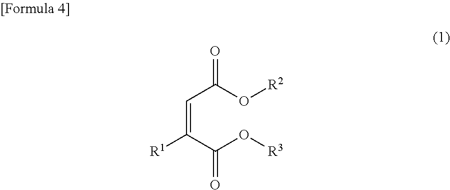

wherein R¹ is the same as R¹ of Formula (2) and R² and R³ are the same as R² and R³ of Formula (3).

The electrolytic solution of the present invention provides a lithium secondary cell which has high capacity, suppresses deterioration in capacity under use upon high-temperature environments, improves cycle characteristics and has long lifespan.

### BRIEF DESCRIPTION OF THE DRAWINGS

FIG. 1 is a view illustrating a configuration of an example of a lithium secondary cell according to the present invention.

FIG. 2 is a view illustrating a configuration of a vehicle using the lithium secondary cell according to the present invention.
a Negative electrode
b Separator
c Positive electrode
d Negative electrode current collector
e Positive electrode current collector
f Positive electrode terminal
g Negative electrode terminal
110 assembled cell (lithium secondary cell)

### DETAILED DESCRIPTION OF THE INVENTION

The lithium secondary cell of the present invention comprises a positive electrode active material layer containing a positive electrode active material, a negative electrode active material layer containing a negative electrode active material and an electrolytic solution in which the positive and negative electrodes are immersed.

### [Negative Electrode Active Material Layer]

Any negative electrode active material layer may be used as long as it contains a negative electrode active material capable of reversibly intercalating and deintercalating lithium ions upon charge/discharge and specifically, it has a configuration in which the negative electrode active material is adhered onto a negative electrode current collector through a binder for the negative electrode.

The negative electrode active material is not particularly limited and is preferably a silicon-based material. Examples of the silicon-based material include silicon, silicon oxide, silicates, silicon compounds of silicon and a transition metal such as nickel, cobalt or the like. The silicon compound is preferably used as the negative electrode active material because it functions to reduce expansion and contraction of the negative electrode active material upon repeated charge/discharge. In addition, according to type of the silicon compound, the silicon compound functions to enable conduction between silicon. In terms of this point, the silicon compound is preferably silicon oxide. Silicon oxide is represented by SiOₓ (0<x<2) and specific examples thereof include SiO, SiO₂ or the like. Silicon oxide is not readily reacted with the electrolytic solution and is stably present in the cell. Silicon oxide may contain Li and is for example represented by SiLi_{y}O_{z} (y>0, 2>z>0). Furthermore, silicon oxide preferably contains at least one element selected from nitrogen, boron and sulfur because deterioration in electric conductivity of the negative electrode active material layer is suppressed and current collection is improved. A content of at least one element selected from nitrogen, boron and sulfur in silicon oxide is preferably 0.1 to 5% by mass because deterioration in energy density of the negative electrode active material layer is suppressed and current collection is improved.

In addition, the silicon-based material preferably contains both elemental silicon and a silicon compound. In particular, the silicon compound is preferably silicon oxide. These substances have different charge/discharge voltages of lithium ions as the negative electrode active material. Specifically, silicon has lower charge/discharge voltages of lithium ions than silicon oxide and the negative electrode active material layer containing the same slowly deintercalates lithium ions according to variation in voltage upon discharge and suppresses rapid volume contraction of the negative electrode active material layer resulting from simultaneous deintercalation of lithium ions at a certain voltage.

The negative electrode active material containing elemental silicon and silicon oxide may be for example prepared by mixing elemental silicon with silicon oxide and sintering the resulting mixture at a high temperature and at a reduced pressure. In addition, when a compound of a transition metal and elemental silicon is used as the silicon compound, the negative electrode active material may be prepared by mixing and melting elemental silicon and the transition metal, or depositing the transition metal on the surface of elemental silicon.

In addition, the negative electrode active material preferably contains a carbon material in terms of superior cycle characteristics, safety and excellent continuous charge characteristics. Examples of the carbon material include coke, acetylene black, mesophase microbeads, graphite or the like. Furthermore, these carbon materials are preferably coated with an organic substance such as pitch and fired or formed an amorphous carbon layer on the surface by CVD. Examples of the organic substance used for coating include coal tar pitches from soft pitch to hard pitch; coal-based heavy oils such as carbonization oils and liquefaction oils; straight heavy oils such as room-temperature residual oils and reduced-pressure residual oils; or decomposition-based heavy oils derived from pyrolysis such as crude oils or naphtha, for example, petroleum-based heavy oils for example ethylene heavy end or the like. In addition, solid residues obtained by distilling these heavy oils at 200 to 400° C. may be ground to a size of 1 to 100 μm. Furthermore, a vinyl chloride resin, a phenol resin, an imide resin or the like may be used for coating the carbon material.

The negative electrode active material preferably contains a carbon material in addition to silicon and silicon oxide in that volume expansion and contraction are reduced upon charge/discharge of the negative electrode active material and conductivity is obtained. Examples of the carbon material used in conjunction with silicon and silicon oxide include graphite, amorphous carbon, diamond carbon, carbon nanotube or the like. Graphite having high crystallinity has superior electrical conductivity, is flat and has excellent adhesion to the current collector. On the other hand, amorphous carbon having low crystallinity undergoes little variation in volume upon charge/discharge, thus suppressing deterioration of the negative electrode active material layer upon charge/discharge. A content of silicon and silicon oxide in the negative electrode active material is preferably not less than 5% by mass and not more than 90% by mass, more preferably not less than 40% by mass and not more than 70% by mass. The content of the carbon material is preferably not less than 2% by mass and not more than 50% by mass, more preferably, not less than 2% by mass and not more than 30% by mass.

When the silicon, silicon oxide and the carbon material are used in particle forms, particles which undergo greater volume variation upon charge/discharge preferably have a smaller diameter because volume variation of the negative electrode active material layer caused by volume variation of the particles is inhibited. Specifically, the average particle diameter of silicon oxide is smaller than the average particle diameter of the carbon material. For example, the average particle diameter of silicon oxide is preferably ½ or less of the average particle diameter of the carbon material. The average particle diameter of silicon is smaller than the average particle diameter of silicon oxide. For example, the average particle diameter of silicon is preferably ½ or less of the average particle diameter of silicon oxide. When the average particle diameter is limited to the range defined above, a secondary cell which greatly reduces volume variation of the negative electrode active material layer because particles which undergo great volume variation upon charge/discharge have a small diameter has excellent balance among energy density, cycle lifespan and efficiency. The average particle diameter of silicon is specifically for example 20 μm or less, preferably, more preferably 15 μm or less, in terms of contact with the current collector.

When the carbon material is contained in conjunction with silicon and silicon oxide as the negative electrode active materials, these substances may be contained as respective particles, but are preferably present as a composite thereof. The composite preferably has a configuration in which silicon oxide is present around silicon clusters and is surface-coated with carbon. In the composite, at least part of silicon oxide preferably has an amorphous structure. It is considered that silicon oxide has an amorphous structure, thereby reducing defects contained in crystal structures or factors caused by non-uniformity of crystal boundaries and the like, suppressing non-uniform volume variation in the composite and facilitating formation of a film on the surface of the carbon material. In addition, formation of a fine powder from the negative electrode active material layer and reaction with the electrolytic solution can be suppressed. The amorphous structure of silicon oxide can be confirmed by X-ray diffraction measurement (general XRD measurement) because inherent peaks observed upon presence of crystal structures broaden.

The composite preferably has a configuration in which silicon is entirely or partially dispersed in silicon oxide. By dispersing at least a part of silicon in silicon oxide, the overall volume expansion of the negative electrode can be further suppressed and decomposition of the electrolytic solution can be also suppressed. The average particle diameter of silicon dispersed in silicon oxide is for example several nanometers to several hundreds of nanometers. The particle diameter may be measured by transmission electron microscopy (TEM). In addition, dispersion of silicon in silicon oxide can be confirmed by using transmission electron microscopy (general TEM observation) in conjunction with energy dispersive X-ray spectrometry (general EDX measurement). Specifically, the dispersion of silicon in silicon oxide can be confirmed by observing a cross-section of a sample, measuring an oxygen concentration of silicon dispersed in silicon oxide and excluding oxygen.

A content of silicon in the composite is preferably not less than 5% by mass and not more than 90% by mass, more preferably not less than 20% by mass and not more than 50% by mass. The content of silicon oxide in the composite is preferably not less than 5% by mass and not more than 90% by mass, more preferably not less than 40% by mass and not more than 70% by mass. The content of the carbon material in the composite is preferably not less than 2% by mass and not more than 50% by mass, more preferably, not less than 2% by mass and not more than 30% by mass.

As a method for preparing a composite having a surface coated with carbon in which silicon is dispersed in silicon oxide having the amorphous structure, for example, silicon oxide, silicon and the carbon material present as particle forms are mixed by mechanical milling. In addition, the composite can be formed by introducing a mixed sinter of silicon and silicon oxide at a high-temperature under a non-oxygen atmosphere and under an organic compound gas atmosphere, or mixing a mixed sinter of silicon and silicon oxide with a precursor resin of carbon at a high-temperature under a non-oxygen atmosphere. The particles used for these methods may have the same average particle diameter described above.

In addition, the composite surface-treated with a silane coupling agent may be used as the negative electrode active material.

Also, the negative electrode active material may contain a metal or metal oxide. The metal is a metal that forms an alloy with lithium and can deintercalate lithium ions from the lithium alloy upon discharge and form the lithium alloy upon charge. Specifically, examples of the metal include aluminum, lead, tin, indium, bismuth, silver, barium, calcium, mercury, palladium, platinum, tellurium, zinc or lanthanum. One or two or more may be selected from these metals. Among the metals, tin is preferred.

Specifically, examples of the metal oxide as the negative electrode active material include aluminum oxide, tin oxide, indium oxide, zinc oxide, lithium oxide or the like. These metals may be used in combination of one, two or more. The metal oxide is preferably used in conjunction with the metal, particularly, the same metal as the metal contained in metal oxide because lithium ions are intercalated and deintercalated at different voltages upon charge/discharge and rapid volume variation of the negative electrode active material layer is suppressed and tin oxide is preferably used in conjunction with tin.

At least parts of these metal oxides preferably partially have an amorphous structure. The metal oxide has an amorphous structure, thereby suppressing formation of a fine powder from the negative electrode active material layer and reaction with the electrolytic solution. The negative electrode active material layer having an amorphous structure, thereby reducing defects contained in crystal structures or factors caused by non-uniformity of crystal boundaries and the like and suppressing non-uniform volume variation. In addition, the metal oxide dispersing a metal therein is preferable.

The particle size of the negative electrode active material is not particularly limited, but is commonly 1 μm or more, preferably 15 μm or more and is commonly 50 μm or less, preferably about 30 μm or less in terms of superior cell characteristics such as first charge/discharge efficiency, rate characteristics and cycle characteristics.

Examples of binders for binding the negative electrode active material include polyvinylidene fluoride (PVdF), vinylidene fluoride-hexafluoropropylene copolymers, vinylidene fluoride-tetrafluoroethylene copolymers, styrene-butadiene copolymer rubbers, polytetrafluoroethylene, polypropylene, polyethylene, polyimide, polyamideimide, polyacrylic acid, polyacrylic acid salts, carboxymethylcellulose, carboxymethylcelluose salts, or the like. These substances may be used alone or in combination of two or more. Among these, polyimide, polyamideimide, polyacrylic acid including lithium salts, sodium salts or potassium salts neutralized with alkali or carboxymethylcelluloses including lithium salts, sodium salts or potassium salts neutralized with alkali are preferred in terms of binding strength. A content of the negative electrode binder used is preferably be 5 to 25 parts by weight with respect to 100 parts by weight of the negative electrode active material in terms of sufficient binding strength and high energy that have a trade off relationship.

Any negative electrode current collector may be used as long as it supports the negative electrode active material layer containing the negative electrode active material integrated by the binder and has conductivity enough to allow a conductive connection with an outer terminal. As a material for the positive electrode current collector, copper, nickel, SUS or the like is preferably used due to electrochemical safety, and copper is preferred in terms of high workability and low cost. Surfaces of the current collector are preferably previously roughened. A shape of the negative electrode current collector may be any of foil, flat, mesh, expanded metal or punching shape such as punching metal.

The negative electrode may be produced by applying a coating solution for the negative electrode active material layer obtained as a slurry from a negative electrode active material and a binder for the negative electrode with a solvent onto a negative electrode current collector and drying the solution. Examples of the coating method include a doctor blade method, a die coater method or the like. In addition, regarding the formation of the negative electrode using a material for the negative electrode active material layer by CVD, sputtering or the like, the material for the negative electrode active material is produced into a sheet electrode by roll-molding or is produced into a pellet electrode by pressing. The negative electrode current collector may be obtained by previously forming the negative electrode active material layer and then forming a thin film of aluminum, nickel or an alloy thereof by deposition, sputtering or the like.

### [Positive Electrode Active Layer]

The positive electrode active layer is not particularly limited and for example, contains a positive electrode active material and has a configuration in which the positive electrode active material is adhered onto the positive electrode current collector by the binder for the positive electrode.

The positive electrode active material deintercalates lithium ions into an electrolytic solution during charging and intercalates the lithium ions from the electrolytic solution during discharging. Examples of the positive electrode active material include lithium manganese oxides having a layered structure such as LiMnO₂ or LixMn₂O₄ (0<x<2), or lithium manganese oxides having a spinel structure; LiCoO₂, LiNiO₂, or those in which a part of the transition metals is substituted by other metal; lithium transition metal oxides such as LiNi_{1/3}Co_{1/3}Mn_{1/3}O₂ in which each transition metal does not exceed half of transition metal atoms; or lithium transition metal oxides in which Li is present in an excess amount than a stoicheiometric composition. In particular, LiαNiβCoγAlδO₂ (1≦α≦1.2, β+γ+δ=1, β≧0.7, γ≦0.2) or LiαNiβCoγMnδO₂ (1≦α≦1.2, β+γ+δ=1, β≧0.6, γ≦0.2) is preferred. The positive electrode active material may be used alone or in combination of two or more.

The positive electrode binder for integrally adhering the positive electrode active material is specifically the same as the negative electrode binder. The positive electrode binder is preferably poly(vinylidene fluoride) in terms of generality and low cost. The amount of the positive electrode binder used is preferably 2 to 10 parts by weight with respect to 100 parts by weight of the positive electrode active material. When the content of the positive electrode binder is 2 parts by weight or more, adhesion between the active materials or between the active material and the current collector is improved and cycle characteristics are enhanced, and when the content is 10 parts by weight or less, a ratio of the active material is increased and positive electrode capacity is enhanced.

The positive electrode active layer may further include a conductive agent to decrease the impedance of the positive electrode active material. As the conductive agent, carbonaceous particulates such as graphite, carbon black or acetylene black may be used.

Any positive electrode current collector may be used as long as it supports the positive electrode active material layer containing the positive electrode active material and has conductivity enough to allow a conductive connection with an external terminal. Specifically, in addition to the same material as that of the negative electrode current collector, aluminum, silver or the like may be used.

The positive electrode may be produced on the positive electrode current collector using a material for the positive electrode active layer containing a positive electrode active material and a binder for the positive electrode. The production method of the positive electrode active layer is the same as that of the negative electrode active material layer.

### [Electrolytic Solution]

The electrolytic solution is prepared by dissolving an electrolytic substance in a non-aqueous organic solvent allowing solubilization of lithium ions. The positive and negative electrodes are immersed in the electrolytic solution, so that these electrodes can intercalate and deintercalate lithium during charge/discharge.

Preferably, the solvent of the electrolytic solution is stable to operation potentials of cells and has a low viscosity to immerse the electrodes regarding use environments of cells. Specifically, examples of the solvent include aprotic organic solvents such as cyclic carbonates such as propylene carbonate (PC), ethylene carbonate (EC), butylene carbonate (BC) or vinylene carbonate (VC); chain carbonates such as dimethyl carbonate (DMC), diethyl carbonate (DEC), ethyl methyl carbonate (EMC) or dipropyl carbonate (DPC); propylene carbonate derivatives; or aliphatic carboxylic acid esters such as methyl formate, methyl acetate or ethyl propionate. These substances may be used alone or in combination of two or more types. Of these, cyclic or chain carbonates such as ethylene carbonate (EC), propylene carbonate (PC), butylene carbonate (BC), vinylene carbonate (VC), dimethyl carbonate (DMC), diethyl carbonate (DEC), methyl ethyl carbonate (MEC) or dipropyl carbonate (DPC) are preferred.

The solvent preferably further contains a fluorinated ether compound. The fluorinated ether compound has high affinity to silicon and improves cycle characteristics in particular, capacity maintenance ratio. The fluorinated ether compound may be a fluorinated chain ether compound in which a part of hydrogen in a non-fluorinated chain ether compound is substituted by fluorine, or a fluorinated cyclic ether compound in which a part of hydrogen in the non-fluorinated cyclic ether compound is substituted by fluorine.

Examples of the non-fluorinated chain ether compound include non-fluorinated chain monoether compounds such as dimethylether, methylethylether, diethylether, methylpropylether, ethylpropylether, dipropylether, methylbutylether, ethylbutylether, propylbutylether, dibutylether, methylpentylether, ethylpentylether, propylpentylether, butylpentylether, or dipentylether; or non-fluorinated chain diether compounds such as 1,2-dimethoxyethane (DME), 1,2-diethoxyethane (DEE), ethoxymethoxyethane (EME), 1,2-dipropoxyethane, propoxyethoxyethane, propoxymethoxyethane, 1,2-dibuthoxyethane, buthoxypropoxyethane, buthoxyethoxyethane, buthoxymethoxyethane, 1,2-dipenthoxyethane, penthoxybuthoxyethane, penthoxypropoxyethane, penthoxyethoxyethane, or penthoxymethoxyethane.

Examples of the non-fluorinated cyclic ether compound include non-fluorinated cyclic monoether compounds such as ethylene oxide, propylene oxide, oxetane, tetrahydrofuran, 2-methyltetrahydrofuran, 3-methyltetrahydrofuran, tetrahydropyran, 2-methyltetrahydropyran, 3-methyltetrahydropyran, or 4-methyltetrahydropyran; or non-fluorinated cyclic diether compounds such as 1,3-dioxolane, 2-methyl-1,3-dioxolane, 4-methyl-1,3-dioxolane, 1,4-dioxane, 2-methyl-1,4-dioxane, 1,3-dioxane, 2-methyl-1,3-dioxane, 4-methyl-1,3-dioxane, 5-methyl-1,3-dioxane, 2,4-dimethyl-1,3-dioxane, or 4-ethyl-1,3-dioxane.

Of these, fluorinated chain ether compounds having superior stability are preferred. The fluorinated chain ether compound is preferably represented by the following formula:

H—(CX¹X²—CX³X⁴)ₙ—CH₂O—CX⁵X⁶—CX⁷X⁸—H

wherein n represents 1, 2, 3 or 4, and X¹ to X⁸ independently represent a fluorine atom or a hydrogen atom, with the proviso that at least one of X¹ to X⁴ represents a fluorine atom and at least one of X⁵ to X⁸ represents a fluorine atom.

The electrolytic substance contained in the electrolyte is preferably a lithium salt. Specifically, examples of the lithium salt include LiPF₆, LiAsF₆, LiAlCl₄, LiClO₄, LiBF₄, LiSbF₆, LiCF₃SO₃, LiC₄F₉SO₃, Li(CF₃SO₂)₃, LiN(CF₃SO₂)₂ or the like.

A concentration of the electrolyte in the electrolytic solution is preferably not less than 0.01 mol/L and not more than 3 mol/L, more preferably, not less than 0.5 mol/L and not more than 1.5 mol/L. When the concentration of the electrolyte is within the range defined above, a cell having high safety, high reliability and low environmental effect may be obtained.

The electrolytic solution contains an ester compound represented by Formula (1).

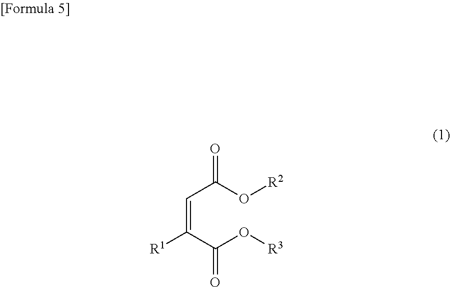

The ester compound represented by Formula (1) is converted into a polymer by polymerization by an unsaturated bond on the surface of the negative electrode active material layer upon charge/discharge of the cell and is made into a film on the negative electrode active material layer. The polymer film permeates lithium ions and suppresses permeation of the solvent of the electrolytic solution, thus suppressing reaction between the negative electrode active material layer and the electrolytic solution and deterioration in cell capacity upon repeated charge/discharge.

In Formula (1), R¹ represents a C2-C12 alkoxy group which may have a substituent or a C2-C12 alkylamino group which may have a substituent and R² and R³ independently represent a hydrogen atom, or a C2-C12 alkyl group which may have a substituent.

The C2-C12 alkoxy group represented by R¹ may be straight or branched, may have a ring and may have an unsaturated bond. Examples of the substituent of the C2-C12 alkoxy group represented by R¹ include a halogen atom such as fluorine atom or chlorine atom, a cyano group, a nitro group, an oxy group or the like.

The C2-C12 alkylamino group represented by R¹ may be linear or branched, may have a ring and may have an unsaturated bond. In particular, for example, the C2-C12 alkylamino group is preferably a C2-C12 dialkylamino group or a ring formed by bonding two alkyl groups. The substituent of the C2-C12 alkylamino group represented by R¹ is for example an oxy group or the like.

R² and R³ independently represent a hydrogen atom, or a C2-C12 alkyl group which may have a substituent. The C2-C12 alkyl group represented by R² and R³ may be linear or branched, but is preferably a methyl group.

Preferred examples of the ester compound represented by Formula (1) include compounds represented by Formulae (5) to (16) in which the C2-C12 alkoxy group represented by R¹ contains 1 to 9 fluorine atoms as substituents.

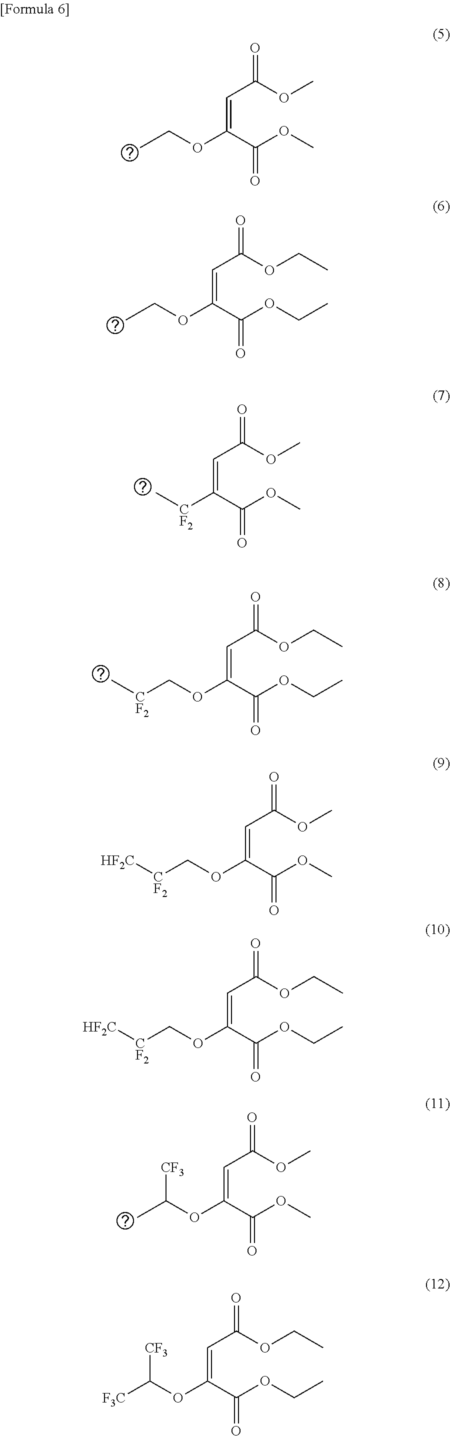

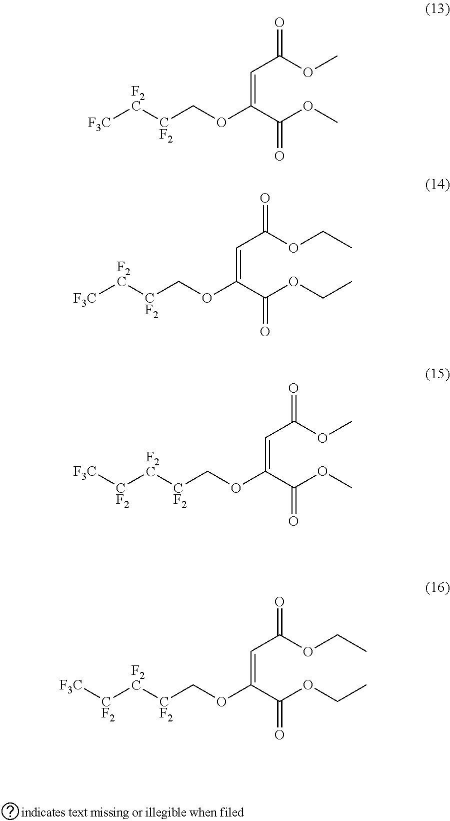

Also, preferred examples of the ester compound represented by Formula (1) include compounds represented by Formulae (17) to (23) in which the C2-C12 alkoxy group represented by R¹ contains a substituent other than a fluorine atom.

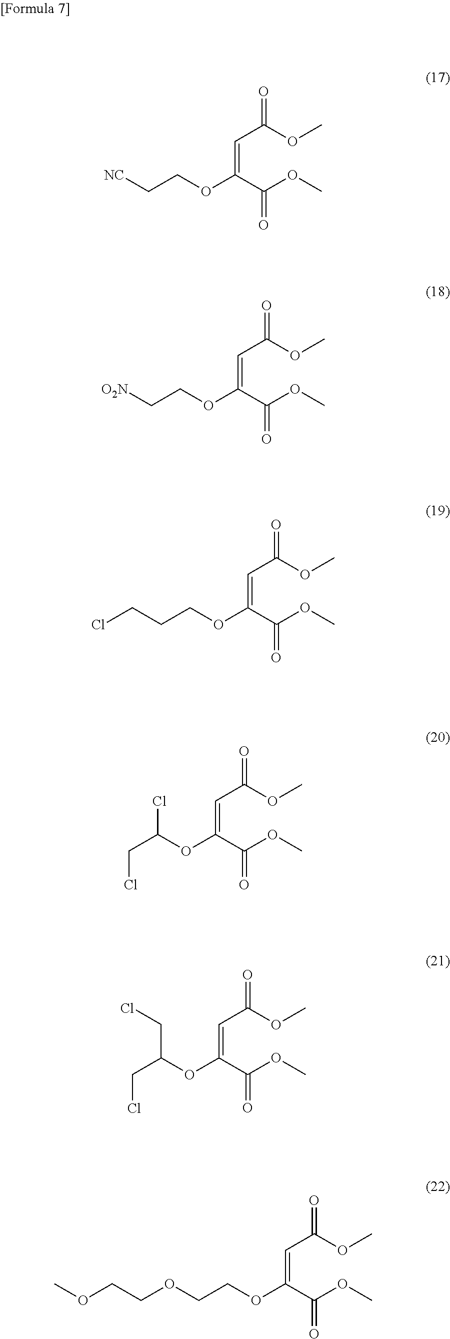

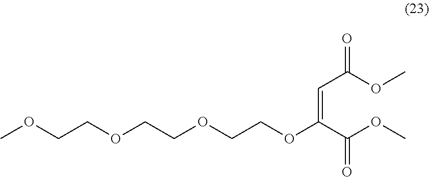

In addition, preferred examples of the ester compound represented by Formula (1) include compounds represented by Formulae (24) to (35) in which R¹ represents a C2-C12 alkylamino group which may have a substituent.

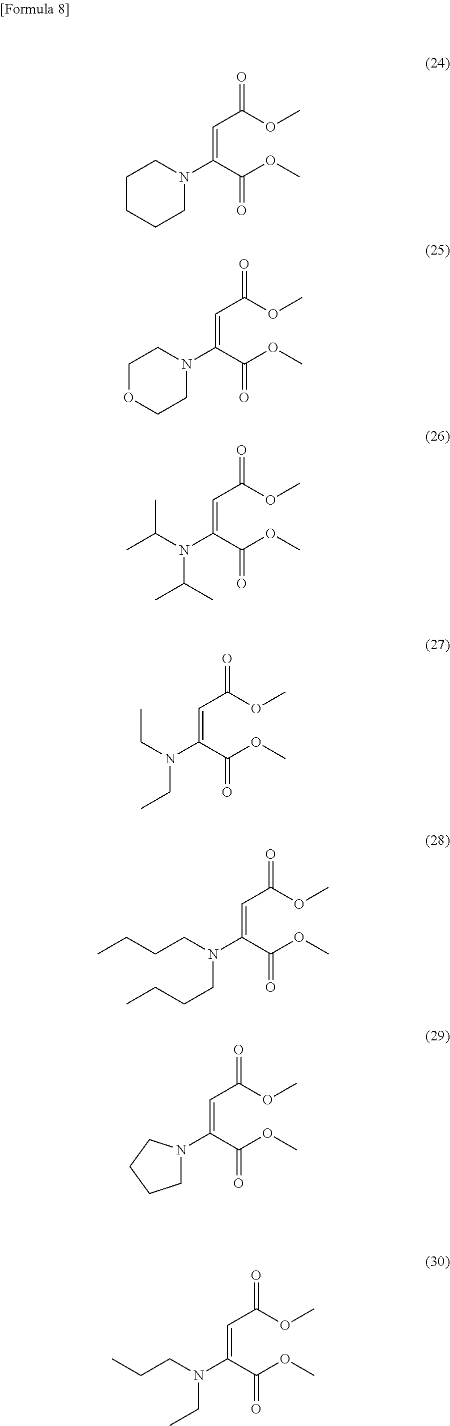

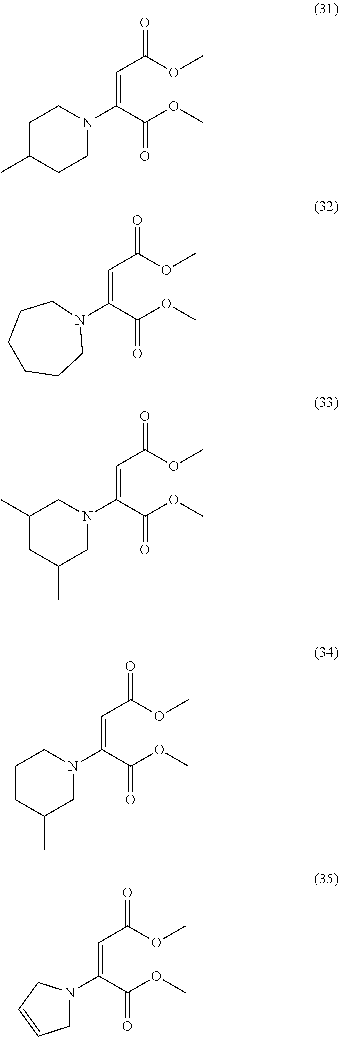

The ester compound represented by Formula (1) may be prepared by a method for preparing an ester compound including reacting an active proton compound represented by the following Formula (2) with an acetylenedicarboxylic acid diester represented by the following Formula (3) to preparing an ester compound represented by Formula (1):

R¹—H (2)

wherein R¹ represents a C2-C12 alkoxy group which may have a substituent or a C2-C12 alkylamino group which may have a substituent; and

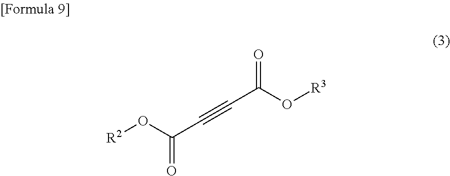

wherein R² and R³ independently represent a hydrogen atom or a C2-C12 alkyl group which may have a substituent;

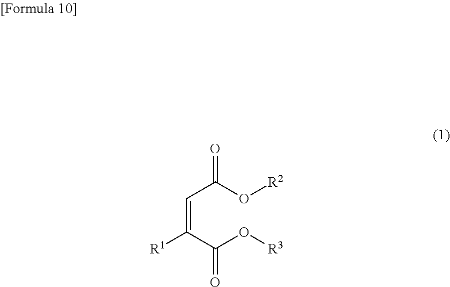

wherein R¹ is the same as R¹ of Formula (2) and R² and R³ are the same as R² and R³ of Formula (3).

In Formula (2), R¹ represents a C2-C12 alkoxy group which may have a substituent or a C2-C12 alkylamino group which may have a substituent. In Formula (2), examples of the C2-C12 alkoxy group which may have a substituent, or the C2-C12 alkylamino group which may have a substituent represented by R¹ is specifically the same as those represented by R¹ in Formula (1) and in Formula (3), examples of the C2-C12 alkyl group which may have a substituent represented by R² and R³ are specifically the same as those represented by R² and R³ of Formula (1).

Examples of the active proton compound represented by Formula (2) include C2-C12 alcohols or C2-C12 secondary amines. The C2-C12 alcohol may be linear or branched, may have a ring, and may have an unsaturated bond and examples of the substituent include halogen atoms such as fluorine atom or chlorine atom, a cyano group, a nitro group, an oxy group or the like. In addition, the C2-C12 secondary amine may be linear or branched, may have a ring and may have an unsaturated bond and the substituent is for example an oxy group or the like.

Specifically, the acetylenedicarboxylic acid diester represented by Formula (3) is for example acetylenedicarboxylic acid dimethyl or the like.

The reaction of the active proton compound represented by Formula (2) with the acetylenedicarboxylic acid diester represented by Formula (3) is for example carried out by cooling to a room temperature or less, for example 0° C., in a solvent such as tetrahydrofuran.

A content of the ester compound represented by Formula (1) in the electrolytic solution is preferably not less than 0.1% by mass and not more than 2.0% by mass. When the concentration of the electrolytic solution is within the range defined above, a film which permeates lithium ions into the negative electrode active material layer and suppresses contact between the electrolytic solution and the negative electrode active material layer.

### [Separator]

Any separator may be used as long as it suppresses a conductive connection between the positive electrode and the negative electrode, allows the penetration of charge carriers, and has durability in the electrolytic solution. Specific materials suitable for the separator may include polyolefin, for example polypropylene or polyethylene based microporous membranes, celluloses, polyethylene terephthalate, polyimide, polyfluorovinylidene or the like. They may be used as a form such as porous film, fabric or nonwoven fabric.

### [Cell Outer Package]

Preferably, the outer package has strength to stably hold the positive electrode, the negative electrode, the separator and the electrolytic solution, is electrochemically stable to these components, and has water-tightness. For example, stainless steel, nickel-plated iron, aluminum, silica, laminate films coated with alumina or the like may be used. As resins for the laminate films, polyethylene, polypropylene, polyethylene terephthalate, or the like may be used. They may be used as a structure of a single layer or two or more layers. A laminate film as the outer package is cheap, as compared to a metal, but is readily deformed due to inner pressure when a gas is generated therein. However, by using the electrolytic solution containing the ester compound, gas generation is suppressed and freedom degree in terms of design of the cell is secured.

### [Secondary Cell]

The secondary cell may have any one of cylindrical, flat winding rectangular, stacked rectangular, coin, flat winding laminate or stacked laminate forms. Such a secondary cell remarkably suppresses gas generation upon charge/discharge, thus suppressing deterioration in the negative electrode active material layer, providing long lifespan, and in particular, suppressing deterioration in the negative electrode active material when used under high-temperature environments. Although stacked laminate-type cells, in which problems of stacked electrodes spread by generated gas and deformation readily occurs, are used under high-temperature environments, the deformation can be suppressed and long lifespan can be obtained.

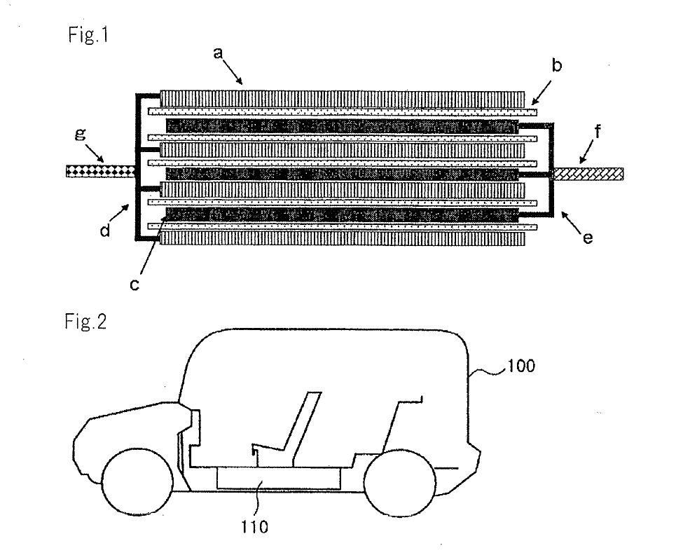

The secondary cell is for example a stacked laminate-type secondary cell shown in FIG. 1. The stacked laminate-type secondary cell has a configuration in which a plurality of negative electrodes (a), each having a negative electrode active material layer mounted on a negative electrode current collector (d) made of a metal such as copper foil, a plurality of positive electrodes (c), each having a positive electrode active layer mounted on a positive electrode current collector (e) made of a metal such as aluminum foil alternately face each other via a separator (b) made of a polypropylene microporous membrane to prevent contact therebetween and are accommodated in a laminate outer package (not shown). The laminate outer package is filled with an electrolytic solution. Each negative electrode (a) is electrically connected to a current collector (d) where an active layer is not formed and each positive electrode (c) is electrically connected to a current collector (e) where an active layer is not formed, and a negative electrode terminal (g) connected to the negative electrode current collector (d) and a positive electrode terminal (f) connected to the positive electrode current collector are exposed to the outside of the laminate outer package and are connected to an external power supply or used device upon charge/discharge.

### [Vehicle]

The lithium secondary cell may be used as a power source for operating motors in vehicles. The vehicle may be any one of electric vehicles or hybrid vehicles.

As an example of the vehicle, an assembled cell including a plurality of lithium secondary cells connected in series or parallel is shown FIG. 2. The vehicle shown in FIG. 2 has a configuration in which the assembled cell 110 of the lithium secondary cell is mounted under the seat of the center of a vehicle body 100.

### EXAMPLE

Hereinafter, the lithium secondary cell of the present invention will be described in detail.

### Synthesis of Ester Compound

### Synthesis Example 1

An ester compound represented by Formula (5) was prepared in accordance with the following synthesis scheme (A).

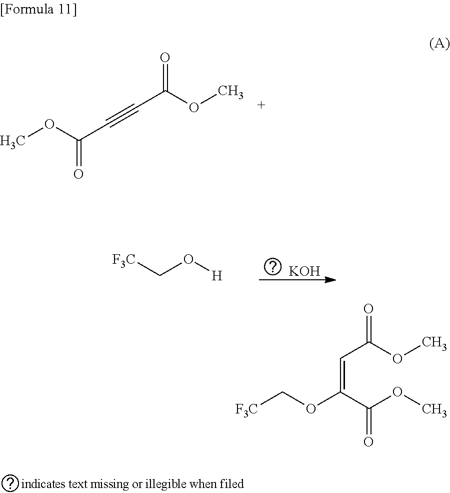

28.5 g of acetylenedicarboxylic acid dimethyl, 40.1 g of 2,2,2-trifluoroethanol and 100 mL of tetrahydrofuran were added to a 500 mL 4-neck flask mounted on an ice bath, followed by cooling to 0° C. 2.24 g of potassium hydroxide was slowly added to the flask and then stirred at 0° C. for 12 hours, the reaction solution was washed with diluted hydrochloric acid, a sodium hydrogen carbonate solution and saturated brine and extracted with diethyl ether and the resulting organic layer was dried in magnesium sulfate. The solvent was removed by distillation using an evaporator and purified by column chromatography to obtain 2-(2,2,2-trifluoroethoxy)maleic acid dimethyl (Formula (5)) with a yield of 51% as a white solid.

¹H NMR(400 MHz, CDCl₃, d): 3.69 (s, 3H, CH₃), 3.87 (s, 3H, CH₃), 4.20 (q, 2H, CH₂, J=8 Hz), 5.27 (s, 1H, C═CH)

### Synthesis Example 2

An ester compound represented by Formula (9) was prepared in accordance with the following synthesis scheme (B).

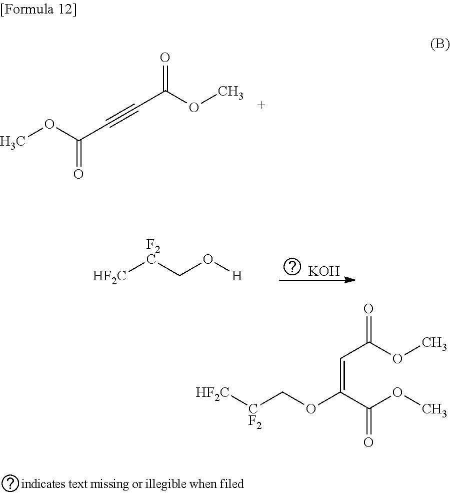

28.5 g of acetylenedicarboxylic acid dimethyl, 53.0 g of 2,2,3,3-tetrafluoroethanol and 100 mL of tetrahydrofuran were added to a 500 mL 4-neck flask mounted on an ice bath, followed by cooling to 0° C. 2.24 g of potassium hydroxide was added to the flask and then stirred at 0° C. for 12 hours, the reaction solution was washed with diluted hydrochloric acid, a sodium hydrogen carbonate solution and saturated brine and extracted with diethyl ether and the resulting organic layer was dried in magnesium sulfate. The solvent was removed by distillation using an evaporator and purified by column chromatography to obtain 2-(2,2,3,3-tetrafluoroethoxy)maleic acid dimethyl (Formula (9)) with a yield of 60% as a white solid.

¹H NMR (400 MHz, CDCl₃, d): 3.68 (s, 3H), 3.85 (s, 3H), 3.89-4.00 (m, 2H), 5.78-6.08 (m, 1H)

### Synthesis Example 3

An ester compound represented by Formula (24) was prepared in accordance with the following synthesis scheme (C).

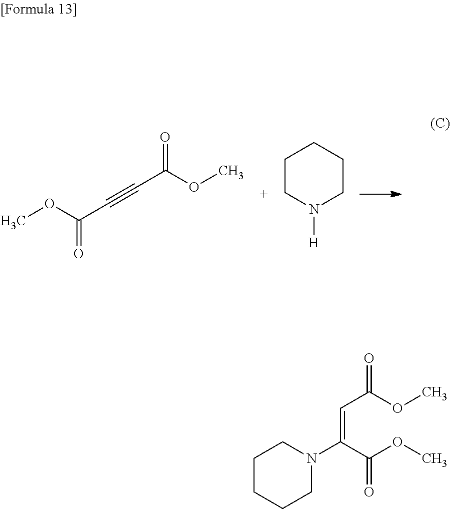

5 g of acetylenedicarboxylic acid dimethyl and 100 mL of tetrahydrofuran were added to a 300 mL 4-neck flask mounted on an ice bath, followed by cooling to 0° C. 25 g of piperidine was slowly added to the flask and then stirred at 0° C. for 2 hours, the reaction solution was washed with diluted hydrochloric acid, a sodium hydrogen carbonate solution and saturated brine and extracted with diethyl ether and the resulting organic layer was dried in magnesium sulfate. The solvent was removed by distillation using an evaporator and purified by column chromatography to obtain 2-piperidylmaleic acid dimethyl (Formula (24)) with a yield of 70% as a white solid.

¹H NMR (400 MHz, CDCl₃, d):1.61 (m, 6H), 3.13 (m, 4H), 3.64 (s, 3H), 3.95 (s, 3H), 4.71 (s, 1H)

### Synthesis Example 4

An ester compound represented by Formula (26) was prepared in accordance with the following synthesis scheme (D).

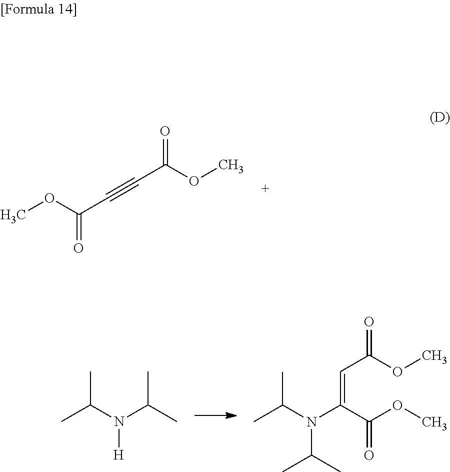

5 g of acetylenedicarboxylic acid dimethyl and 100 mL of tetrahydrofuran were added to a 300 mL 4-neck flask mounted on an ice bath, followed by cooling to 0° C. 25 g of diisopropylamine was slowly added to the flask and then stirred at 0° C. for 2 hours, the reaction solution was washed with diluted hydrochloric acid, a sodium hydrogen carbonate solution and saturated brine, and extracted with diethyl ether and the resulting organic layer was dried in magnesium sulfate. The solvent was removed by distillation using an evaporator and purified by column chromatography to obtain 2-(diisopropylamino)maleic acid dimethyl (Formula (26)) with a yield of 75% as a white solid.

¹H NMR (400 MHz, CDCl₃, d):1.25 (m, 12H), 3.46 (m, 6H), 3.91 (m, 2H), 4.74 (s, 1H)

### Example 1

### Production of Negative Electrode

Silicon having an average particle size of 5 μm and graphite having an average particle size of 30 μm were weighed as negative electrode active materials at a weight ratio of 90:10 and the materials were mixed by mechanical milling for 24 hours to obtain a negative electrode active material. The negative electrode active material (average particle size D50=5 nm) and polyimide (U Varnish® A: produced by Ube Industries, Ltd.) as a binder for the negative electrode were weighed at a weight ratio of 85:15 and were mixed with n-methyl pyrrolidone to obtain a negative electrode slurry. The negative electrode slurry was applied to a copper foil with a thickness of 10 nm, dried and thermally treated under a nitrogen atmosphere at 300° C. to produce a negative electrode.

### [Production of Positive Electrode]

Nickel oxide lithium (LiNi_{0.80}Co_{0.15}Al_{0.15}O₂) as a positive electrode active material, carbon black as a conductive agent and poly(vinylidene fluoride) as a binder for the positive electrode were weighed at a weight ratio of 90:5:5 and mixed with n-methyl pyrrolidone to obtain a positive electrode slurry. The positive electrode slurry was applied to an aluminum foil with a thickness of 20 nm, dried and pressed to produce a positive electrode.

### [Preparation of Electrolytic Solution]

0.2% by mass of the ester compound represented by Formula (5) was mixed with a carbonate non-aqueous solvent consisting of EC/DEC=30/70 dissolving LiPF₆ as an electrolyte at a concentration of 1 mol/L to obtain an electrolytic solution.

### [Production of Lithium Secondary Cell]

Three layers of the obtained positive electrode and four layers of the obtain electrode negative electrode were alternately stacked such that a polypropylene porous film as a separator was interposed between the positive electrode and the negative electrode. Ends of the positive electrode current collectors not coated with the positive electrode active layers and ends of the negative electrode current collectors not coated with the negative electrode active material layers were respectively welded and a positive electrode terminal made of aluminum and a negative electrode terminal made of nickel were respectively welded to the welded ends to obtain an electrode device having a flat stack structure.

The electrode device was covered with an aluminum laminate film as an outer package, an electrolytic solution was filled therein and sealed while decreasing the pressure to 0.1 atm to produce a secondary cell.

### [Evaluation of Charge/Discharge Cycle Characteristics]

The high-temperature cycle characteristics of the produced lithium secondary cell were measured as follows. The secondary cell was charged/discharged 50 times in a 60° C. constant-temperature bath at a voltage of 2.5V to 4.1V and discharge capacity thereof was measured. A ratio D50/D5 (unit: %) of the 50^{th} cycle discharge capacity (D50) to the 5^{th} cycle discharge capacity (D5) was calculated and defined as a maintenance ratio. In addition, a ratio V50/V5 (unit: %) of the 50^{th} cycle cell volume (V50) to the 5^{th} cycle cell volume (V5) was calculated and defined as a swelling ratio. Results are shown in Table 1.

A maintenance ratio of 75% or more is evaluated as “A”, a maintenance ratio of not less than 50% and less than 75% is evaluated as “B”, a maintenance ratio of not less than 25% and less than 50% is evaluated as “C” and a maintenance ratio of less than 25% is evaluated as “D”. A swelling ratio of less than 4% is evaluated as “A”, a swelling ratio of not less than 4% and less than 10% is evaluated as “B”, a maintenance ratio of not less than 10% and less than 20% is evaluated as “C” and a maintenance ratio of not less than 20% is evaluated as “D”. Results are shown in Table 1.

### Examples 2 to 4

Secondary cells were produced in the same manner as in Example 1, except that the ester compound shown in Table 1 was used instead of the ester compound represented by Formula (5) and cycle characteristics were evaluated. Results are shown in Table 1.

### Examples 5 to 8

Secondary cells were produced in the same manner as in Example 1, except that polyamideimide (PAI)(PYROMAX® produced by Toyobo Co., Ltd.) was used as the negative electrode binder instead of polyimide and the ester compound shown in Table 1 was used instead of the ester compound represented by Formula (5) and cycle characteristics were evaluated. Results are shown in Table 1.

### Examples 9 to 12

A negative electrode active material was obtained in the same manner as in Example 1, using silicon and graphite added amorphous silicon oxide (SiOₓ, 0<x≦2) as the negative electrode active materials instead of silicon and graphite at a weight ratio of silicon, amorphous silicon oxide and graphite of 29:61:10. The obtained negative electrode active materials were present as particles having an average particle size (D50) of 5 μm in which silicon was dispersed in silicon oxide. A secondary cell was produced in the same manner as in Example 1, except that the ester compound shown in Table 1 was used instead of the ester compound represented by Formula (5) using the obtained negative electrode active material and cycle characteristics were evaluated. Results are shown in Table 1.

### Examples 13 to 16

Secondary cells were produced in the same manner as in Example 1, except that the substance used in Example 9 was used as the negative electrode active material, polyamideimide (PAI, PYROMAX® produced by Toyobo Co., Ltd.) was used as the negative electrode binder instead of polyimide and the ester compound shown in Table 1 was used instead of the ester compound represented by Formula (5) and cycle characteristics were evaluated. Results are shown in Table 1.

TABLE 1

| | Negative | | | | | | | |
|---|---|---|---|---|---|---|---|---|
| | electrode | | | | | | | |
| | active | Binder | | | Maintenance | | Swelling | |
| | material | for | | Dispersion | ratio | | ratio | |
| | Si/SiOₓ/C | negative | Ester | of Si in | | Evalua- | | Evalua- |
| Examples | (wt ratio) | electrode | compound | SiOₓ | % | tion | % | tion |
| Example 1 | 90/0/10 | PI | Formula (5) | Absence | 60 | B | 5 | B |
| Example 2 | 90/0/10 | PI | Formula (9) | Absence | 61 | B | 4 | B |
| Example 3 | 90/0/10 | PI | Formula (24) | Absence | 62 | B | 5 | B |
| Example 4 | 90/0/10 | PI | Formula (26) | Absence | 63 | B | 7 | B |
| Example 5 | 90/0/10 | PAI | Formula (5) | Absence | 68 | B | 5 | B |
| Example 6 | 90/0/10 | PAI | Formula (9) | Absence | 67 | B | 6 | B |
| Example 7 | 90/0/10 | PAI | Formula (24) | Absence | 69 | B | 7 | B |
| Example 8 | 90/0/10 | PAI | Formula (26) | Absence | 67 | B | 5 | B |
| Example 9 | 29/61/10 | PI | Formula (5) | Presence | 70 | B | 5 | B |
| Example 10 | 29/61/10 | PI | Formula (9) | Presence | 73 | B | 6 | B |
| Example 11 | 29/61/10 | PI | Formula (24) | Presence | 67 | B | 6 | B |
| Example 12 | 29/61/10 | PI | Formula (26) | Presence | 70 | B | 6 | B |
| Example 13 | 29/61/10 | PAI | Formula (5) | Presence | 71 | B | 5 | B |
| Example 14 | 29/61/10 | PAI | Formula (9) | Presence | 71 | B | 5 | B |
| Example 15 | 29/61/10 | PAI | Formula (24) | Presence | 74 | B | 7 | B |
| Example 16 | 29/61/10 | PAI | Formula (26) | Presence | 75 | B | 7 | B |

### Comparative Example 1

Secondary cells were produced in the same manner as in Example 1, except that the ester compound represented by Formula (5) was not added to the electrolytic solution, and cycle characteristics were evaluated. Results are shown in Table 2.

### Comparative Examples 2 to 4

Secondary cells were produced in the same manner as in Example 1, except that succinic anhydride, phthalic anhydride and benzoic anhydride were respectively used instead of the ester compound represented by Formula (5) and cycle characteristics were evaluated. Results are shown in Table 2.

### Comparative Example 5

A secondary cell was produced in the same manner as in Example 5, except that the ester compound represented by Formula (5) was not added to the electrolytic solution and cycle characteristics were evaluated. Results are shown in Table 2.

### Comparative Examples 6 to 8

Secondary cells were produced in the same manner as in Example 5, except that succinic anhydride, phthalic anhydride and benzoic anhydride were respectively used instead of the ester compound represented by Formula (5) and cycle characteristics were evaluated. Results are shown in Table 2.

### Comparative Example 9

A secondary cell was produced in the same manner as in Example 13, except that the ester compound represented by Formula (5) was not added to the electrolyte and cycle characteristics were evaluated. Results are shown in Table 2.

### Comparative Examples 10 to 12

Secondary cells were produced in the same manner as in Example 13, except that succinic anhydride, phthalic anhydride and benzoic anhydride were respectively used instead of the ester compound represented by Formula (5) and cycle characteristics were evaluated. Results are shown in Table 3.

### Comparative Example 13

A secondary cell was produced in the same manner as in Example 9, except that the ester compound represented by Formula (5) was not added to the electrolytic solution and cycle characteristics were evaluated. Results are shown in Table 2.

### Comparative Examples 14 to 16

Secondary cells were produced in the same manner as in Example 9, except that succinic anhydride, phthalic anhydride and benzoic anhydride were respectively used instead of the ester compound represented by Formula (5) and cycle characteristics were evaluated. Results are shown in Table 2.

TABLE 2

| | Negative | | | | | | | |
|---|---|---|---|---|---|---|---|---|
| | electrode | | | | | | | |
| | active | Binder | | | Maintenance | | Swelling | |
| | material | for | | Dispersion | ratio | | ratio | |
| | Si/SiOₓ/C | negative | Acid | of Si in | | Evalua- | | Evalua- |
| Examples | (wt ratio) | electrode | anhydride | SiOₓ | % | tion | % | tion |
| Comparative | 90/0/10 | PI | Absence | Absence | 41 | C | 23 | D |
| Example 1 | | | | | | | | |
| Comparative | 90/0/10 | PI | Succinic | Absence | 44 | C | 20 | C |
| Example 2 | | | anhydride | | | | | |
| Comparative | 90/0/10 | PI | Phthalic | Absence | 43 | C | 19 | C |
| Example 3 | | | anhydride | | | | | |
| Comparative | 90/0/10 | PI | Benzoic | Absence | 44 | C | 18 | C |
| Example 4 | | | anhydride | | | | | |
| Comparative | 90/0/10 | PAI | Absence | Absence | 40 | C | 19 | C |
| Example 5 | | | | | | | | |
| Comparative | 90/0/10 | PAI | Succinic | Absence | 43 | C | 25 | D |
| Example 6 | | | anhydride | | | | | |
| Comparative | 90/0/10 | PAI | Phthalic | Absence | 45 | C | 19 | C |
| Example 7 | | | anhydride | | | | | |
| Comparative | 90/0/10 | PAI | Benzoic | Absence | 44 | C | 18 | C |
| Example 8 | | | anhydride | | | | | |
| Comparative | 29/61/10 | PAI | Absence | Absence | 42 | C | 24 | D |
| Example 9 | | | | | | | | |
| Comparative | 29/61/10 | PAI | Succinic | Absence | 44 | C | 18 | C |
| Example 10 | | | anhydride | | | | | |
| Comparative | 29/61/10 | PAI | Phthalic | Absence | 43 | C | 17 | C |
| Example 11 | | | anhydride | | | | | |
| Comparative | 29/61/10 | PAI | Benzoic | Absence | 44 | C | 18 | C |
| Example 12 | | | anhydride | | | | | |
| Comparative | 29/61/10 | PI | Absence | Presence | 42 | C | 27 | D |
| Example 13 | | | | | | | | |
| Comparative | 29/61/10 | PI | Succinic | Presence | 45 | C | 19 | C |
| Example 14 | | | anhydride | | | | | |
| Comparative | 29/61/10 | PI | Phthalic | Presence | 44 | C | 18 | C |
| Example 15 | | | anhydride | | | | | |
| Comparative | 29/61/10 | PI | Benzoic | Presence | 43 | C | 17 | C |
| Example 16 | | | anhydride | | | | | |

As can be seen from the results, the secondary cells of Examples exhibited lower swelling ratios at 60° C. as compared to that of Comparative Examples and the lithium secondary cells of the present invention exhibited superior cycle characteristics.

This application incorporates the full disclosure of JP Patent Application No. 2012-128833 filed on Jun. 6, 2012 herein by reference.

The present invention is applicable to all of industrial fields that require power source and industrial fields related to transmission, storage and supply of electrical energy. Specifically, the present invention is applicable to power sources for mobile devices such as cellular phones and notebook computers and the like.

1. An electrolytic solution comprising an ester compound represented by the following Formula (1): 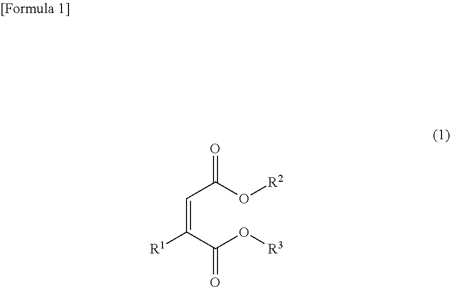
wherein R¹ represents a C2-C12 alkoxy group which may have a substituent or a C2-C12 alkylamino group which may have a substituent, and R² and R³ independently represent a hydrogen atom or a C2-C12 alkyl group which may have a substituent.

2. The electrolytic solution according to claim 1, wherein in Formula (1), R¹ represents a C2-C12 alkoxy group substituted by 1 to 9 fluorine atoms.

3. The electrolytic solution according to claim 1, wherein in Formula (1), R¹ represents a C2-C12 dialkylamino group which may have a substituent.

4. The electrolytic solution according to claim 1, wherein the ester compound represented by Formula (1) is contained in an amount of not less than 0.1% by mass and not more than 2.0% by mass.

5. A lithium secondary cell comprising a positive electrode active material layer containing a positive electrode active material, a negative electrode active material layer containing a negative electrode active material and an electrolytic solution for immersing the positive and negative electrode active material layers,
wherein the electrolytic solution comprises an ester compound represented by Formula (1): 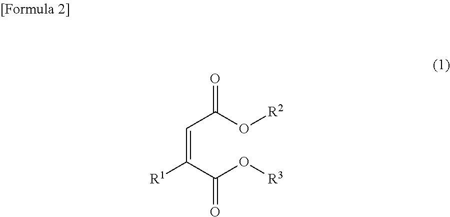
wherein R¹ represents a C2-C12 alkoxy group which may have a substituent or a C2-C12 alkylamino group which may have a substituent, and R² and R³ independently represent a hydrogen atom or a C2-C12 alkyl group which may have a substituent.

6. The lithium secondary cell according to claim 5, wherein the negative electrode active material comprises a silicon-based material.

7. The lithium secondary cell according to claim 5, wherein the negative electrode active material layer comprises a carbon-based material.

8. The lithium secondary cell according to claim 6, wherein the silicon-based material comprises silicon oxide and at least a part of the silicon oxide has an amorphous structure.

9. A method for preparing an ester compound comprising reacting an active proton compound represented Formula (2) with an acetylenedicarboxylic acid diester represented by the following Formula (3) to preparing an ester compound represented by Formula (1):
R¹—H (2)
wherein R¹ represents a C2-C12 alkoxy group which may have a substituent or a C2-C12 alkylamino group which may have a substituent; 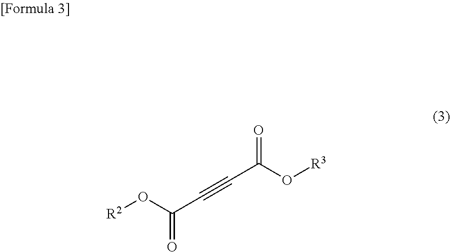
wherein R² and R³ independently represent a hydrogen atom, or a C2-C12 alkyl group which may have a substituent; and 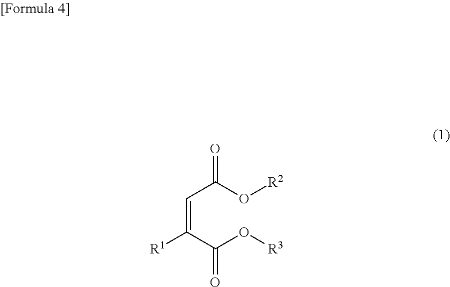
wherein R¹ is the same as R¹ of Formula (2) and R² and R³ are the same as R² and R³ of Formula (3).

10. The method according to claim 9, wherein the active proton compound is alcohol or secondary amine.
